(19) United States
(12) Patent Application Publication
(10) Pub. No.: US 2025/0251407 A1
(43) Pub. Date: Aug. 7, 2025

(54) MONOCLONAL ANTIBODY COMPOSITION FOR QUANTITATIVE DETECTION OF AMYLOID IN HUMAN BODY FLUID AND USES

(71) Applicant: BEIJING JIAOTONG UNIVERSITY, Beijing (CN)

(72) Inventors: Ying ZHANG, Beijing (CN); Jinsheng HE, Beijing (CN); Zidi YAN, Beijing (CN); Tao HONG, Beijing (CN)

(73) Assignee: BEIJING JIAOTONG UNIVERSITY, Beijing (CN)

(21) Appl. No.: 19/186,692

(22) Filed: Apr. 23, 2025

Related U.S. Application Data
(63) Continuation of application No. PCT/CN2024/098036, filed on Jun. 7, 2024.

(30) Foreign Application Priority Data
Jun. 19, 2023 (CN) 202310726310.0

(51) Int. Cl. G01N 33/68; C07K 16/18

(52) U.S. Cl. CPC G01N 33/6896; C07K 16/18; C07K 2317/33; G01N 2333/4709; G01N 2800/2821

(57) ABSTRACT

A monoclonal antibody composition comprises a capture antibody AB7G and a detection antibody AB11A2 for using in preparation of a kit for quantitative detection of amyloid in human body fluids, and the antibodies are monoclonal antibodies secreted by cultured hybridoma cells. The kit specifically recognizes an Aβ42 oligomer, with a linear detection range of 3.9-125 pg/mL, and a lowest detection limit of 7.8 pg/mL. A core technique for kit assembly lies in that the monoclonal antibody AB7G is fixed on a microplate to serve as the capture antibody, and the monoclonal antibody AB11A2 is labeled and then diluted at 1:2000 to serve as the detection antibody. Meanwhile, the present invention relates to heavy and light chain variable region genes of the monoclonal antibodies AB7G and AB11A2 and peptides encoded thereby.

### CROSS-REFERENCE TO RELATED APPLICATIONS

This application is the U.S. continuation application of International Application No. PCT/CN 2024/098036 filed on 7 Jun. 2024 which designated the U.S. and claims priority to Chinese Application No. CN 202310726310.0 filed on 19 Jun. 2023, the entire contents of each of which are hereby incorporated by reference.

### REFERENCE TO AN ELECTRONIC SEQUENCE LISTING

The contents of the electronic sequence listing (250101.xml; Size: 30,602 bytes; and Date of Creation: Feb. 14, 2025) is herein incorporated by reference in its entirety.

### BACKGROUND OF THE INVENTION

### 1. Technical Field

The present invention relates to the technical field of genetic engineering, in particular to a monoclonal antibody composition for quantitative detection of amyloid in human body fluids and uses, a monoclonal antibody against human β amyloid peptide (Aβ42), hybridoma cell strains, and uses thereof for quantitative detection of an Aβ42 oligomer in human body fluids, and for preparation of a reagent and medicament for prevention, diagnosis or treatment of Aβ-driven amyloidosis.

### 2. Description of Related Art

Alzheimer's disease (AD) is an irreversible progressive, neurodegenerative disease featured with impaired memory and cognitive disorder. Amyloid β (Aβ) oligomers in the brain of a patient with AD show an early pathological change of the disease. Soluble Aβ42 oligomers, as main toxic substances in the early stage of AD, can cause cognitive impairment in the patient in the early stage of this disease. Experiments have shown that the toxicity of the Aβ42 oligomers mainly lies in the damage to neurons, which eventually causes neuronal death. Therefore, the study on the structure of Aβ42 oligomers and the damage of the β42 oligomers to neurons is of great significance for in-depth understanding of the role of Aβ42 oligomers in the pathogenesis of AD.

The level of soluble Aβ oligomers (sOABs) undergoes pathological changes in body fluids such as cerebrospinal fluid and blood in patients in the early stage of AD. Therefore, detection of the level of OAB can be used for early diagnosis of AD and evaluation of medicament efficacy. In spite of Aβ42 detection kits available at present, the Aβ42 oligomers (sOAβs) cannot not be specifically and quantitatively detected in body fluids. The soluble Aβ42 oligomers are known to be the most important early pathogenic factor of AD and show the highest toxicity to neurons. At present, there is still a lack of a method for quantitative detection of Aβ42 oligomers in human fluids for early diagnostic purposes.

### BRIEF SUMMARY OF THE INVENTION

An object of the present invention is to provide a monoclonal antibody against human amyloid-β Aβ42 and its uses for quantitative detection of an Aβ42 oligomer in human body fluids and for preparation of a reagent and medicament for prevention, diagnosis or treatment of Aβ-driven amyloidosis.

To achieve the above object, the present invention employs a technical solution as follows.

In an aspect, the present invention provides a monoclonal antibody composition for quantitative detection of amyloid in human body fluids, comprising a monoclonal antibody AB7G, or a fragment thereof, against human Aβ42, and a monoclonal antibody AB11A2 or a fragment thereof for detecting an Aβ42 monomer or an aggregate thereof in combination with the monoclonal antibody AB7G or the fragment thereof, wherein the monoclonal antibody AB7G or the fragment thereof is able to specifically bind to an Aβ42 monomer and an Aβ42 aggregate; an amino acid sequence of a heavy chain variable region of the monoclonal antibody AB7G or the fragment thereof is as set forth in SEQ ID NO: 2; and an amino acid sequence of a light chain variable region of the monoclonal antibody AB7G or the fragment thereof is as set forth in SEQ ID NO: 4; an amino acid sequence of a heavy chain of the monoclonal antibody AB7G is as set forth in SEQ ID NO: 19; and an amino acid sequence of a light chain of the monoclonal antibody AB7G is as set forth in SEQ ID NO: 21.

An amino acid sequence of a heavy chain variable region of the monoclonal antibody AB11A2 or the fragment thereof is as set forth in SEQ ID NO: 6; and an amino acid sequence of a light chain variable region of the monoclonal antibody AB11A2 or the fragment thereof is as set forth in SEQ ID NO: 8; an amino acid sequence of a heavy chain of the monoclonal antibody AB11A2 is as set forth in SEQ ID NO: 23; and an amino acid sequence of a light chain of the monoclonal antibody AB11A2 is as set forth in SEQ ID NO: 25.

In a second aspect, the present invention provides a nucleic acid encoding the monoclonal antibody AB7G or the fragment thereof as defined above. A nucleic acid sequence encoding the heavy chain variable region of the monoclonal antibody AB7G or the fragment thereof is as set forth in SEQ ID NO: 1; and a nucleic acid sequence encoding the light chain variable region of the monoclonal antibody AB7G or the fragment thereof is as set forth in SEQ ID NO: 3. A nucleic acid sequence encoding the heavy chain of the monoclonal antibody AB7G is as set forth in SEQ ID NO: 18; and a nucleic acid sequence encoding the light chain of the monoclonal antibody AB7G is as set forth in SEQ ID NO: 20.

In a third aspect, the present invention provides a nucleic acid encoding the monoclonal antibody AB11A2 or the fragment thereof as defined above. A nucleic acid sequence encoding the heavy chain variable region of the monoclonal antibody AB11A2 or the fragment thereof is as set forth in SEQ ID NO: 5; and a nucleic acid sequence encoding the light chain variable region of the monoclonal antibody AB11A2 or the fragment thereof is as set forth in SEQ ID NO: 7. A nucleic acid sequence encoding the heavy chain of the monoclonal antibody AB11A2 is as set forth in SEQ ID NO: 22; and a nucleic acid sequence encoding the light chain of the monoclonal antibody AB11A2 is as set forth in SEQ ID NO: 24.

In a fourth aspect, the present invention provides a biological material comprising the nucleic acid encoding the monoclonal antibody AB7G or the fragment thereof as defined above, wherein the biological material is an expression cassette, a vector, a transposon or a host cell.

In a fifth aspect, the present invention provides a biological material comprising the nucleic acid encoding the monoclonal antibody AB11A2 or the fragment thereof as defined above, wherein the biological material is an expression cassette, a vector, a transposon or a host cell.

In a sixth aspect, the present invention provides a complex or conjugate, comprising or being prepared from the monoclonal antibody AB7G or the fragment thereof. The complex or conjugate is derived by chemically or biologically labeling the monoclonal antibody AB7G or the fragment thereof; and the conjugate is derived by conjugating the monoclonal antibody AB7G or the fragment thereof or the complex to a solid or semi-solid medium.

In a seventh aspect, the present invention provides a complex or conjugate, comprising or being prepared from the monoclonal antibody AB11A2 or the fragment thereof. The complex is derived by chemically or biologically labeling the monoclonal antibody AB11A2 or the fragment thereof; and the conjugate is derived by conjugating the monoclonal antibody AB11A2 or the fragment thereof or the complex to a solid or semi-solid medium.

In an eighth aspect, the present invention provides use of the monoclonal antibody AB7G or the fragment thereof, or the nucleic acid encoding the monoclonal antibody AB7G or the fragment thereof, or the monoclonal antibody AB11A2 or the fragment thereof, or the nucleic acid encoding the monoclonal antibody AB11A2 or the fragment thereof, or the biological material, or the complex or conjugate, as defined above, for any one of:
(1) preparation of a reagent and medicament for prevention, diagnosis or treatment of an Aβ42-driven amyloidosis;
(2) preparation of a product for mitigation of polymeric toxicity of the Aβ42 or for control of development of the Aβ42-driven amyloidosis;
(3) preparation of a product for inhibition of formation of the Aβ42 monomer or an aggregate, fibril or corpus fibrosum of the Aβ42 monomer;
(4) preparation of a product for reduction of accumulation of the Aβ42 aggregate; and
(5) preparation of a reagent or kit for detection of the Aβ42 monomer, the Aβ42 aggregate, and an Aβ42 antibody.

In a ninth aspect, the present invention provides a medicament, comprising or being prepared from the monoclonal antibody AB7G or the fragment thereof, or further comprising the monoclonal antibody AB11A2 or the fragment thereof, wherein the medicament is for use in any one of:
(1) prevention, diagnosis or treatment of an Aβ42-driven amyloidosis;
(2) mitigation of polymeric toxicity of the Aβ42 or control of development of the Aβ42-driven amyloidosis;
(3) inhibition of formation of the Aβ42 monomer or an aggregate, fibril or corpus fibrosum of the Aβ42; and
(4) reduction of accumulation of the aggregate, fibril or corpus fibrosum of the Aβ42.

The Aβ42-driven amyloidosis in the present invention is a disease manifested as a result of abnormal Aβ42 metabolism, and comprises AD or complicated AD, etc.

In a tenth aspect, the present invention provides a kit for specific quantitative detection of an Aβ42 monomer or an aggregate thereof in human body fluids, comprising: the monoclonal antibody AB7G or the fragment thereof as defined above, for capturing the Aβ42 monomer or the aggregate thereof; and the monoclonal antibody A B 11A2 or the fragment thereof as defined above, for detecting the Aβ42 monomer or the aggregate thereof. The monoclonal antibody AB7G or the fragment thereof is immobilized on a microplate, and the monoclonal antibody AB11A2 or the fragment thereof is labeled with biotin.

In an eleventh aspect, the present invention provides use of the nucleic acid encoding the monoclonal antibody AB7G or the fragment thereof or the biological material, as defined above, for preparation of a monoclonal antibody against human amyloid.

In a twelfth aspect, the present invention provides use of the nucleic acid encoding the monoclonal antibody AB11A2 or the fragment thereof or the biological material, as defined above, for preparation of a monoclonal antibody against human amyloid.

In a thirteenth aspect, the present invention provides use of the nucleic acid encoding the monoclonal antibody AB7G or the fragment thereof as defined above for construction of a genetically engineered monoclonal antibody expression system.

In a fourteenth aspect, the present invention provides use of the nucleic acid encoding the monoclonal antibody AB11A2 or the fragment thereof as defined above for construction of a genetically engineered monoclonal antibody expression system.

The monoclonal antibodies AB7G and AB11A2 in the present invention are monoclonal antibodies secreted by cultured hybridoma cells AB7G and AB11A2, respectively. The hybridoma cells AB7G and AB11A2 are collected in the China Center for Type Culture Collection, with Accession Numbers of CCTCC C201256 and CCTCC C2012130 respectively.

The detailed biological material collection information is as follows:

### (1) Culture Name: Hybridoma Cell Strain AB7G

Accession Number: CCTCC-C201256
Collection Institute: China Center for Type Culture Collection (CCTCC)
Collection Date: Apr. 13, 2012

### (2) Culture Name: Hybridoma Cell Strain AB11A2

Accession Number: CCTCC-C2012130
Collection Institute: China Center for Type Culture Collection (CCTCC)
Collection Date: Sep. 11, 2012

Beneficial effects of the present invention: the monoclonal antibodies of the capture and detection antibodies for kit assembly are specifically matched, with the detection sensitivity up to 7.8 pg/mL for the calibration material Aβ42 oligomer of 17-95 kD, and the specificity is high; and the detection kit for amyloid in human body fluids can be used for detecting Aβ42 oligomers of 17-95 kD in cerebrospinal fluid and blood at the same time, showing the application prospect of early diagnosis of AD, and only 5-15 μL of the sample is needed.

Additional aspects and advantages of the present invention will be partially given in the description below, and these aspects and advantages will become apparent from the description below or will be learned by practicing the invention.

### BRIEF DESCRIPTION OF THE DRAWINGS

To describe the technical solutions in the embodiments of the present invention more clearly, the following briefly introduces the accompanying drawings required for describing the embodiments. Obviously, the accompanying drawings in the following description show merely some embodiments of the present invention, and those ordinarily skilled in the art may still derive other drawings from these accompanying drawings without creative labor.

FIG. 1 is a schematic diagram of the calibration curve of a kit according to an embodiment of the present invention;

FIG. 2 is a schematic diagram of identification result of a calibration material Aβ42 oligomer according to an embodiment of the present invention;

FIG. 3 is a schematic diagram of the screening of antibody matching methods according to an embodiment of the present invention;

FIG. 4 is a schematic diagram of the optimization process of a calibration curve according to an embodiment of the present invention;

FIG. 5 is a schematic diagram of the detection specificity of the kit according to an embodiment of the present invention;

FIG. 6 is a schematic diagram of the detection of a sample of the extract of brain tissues according to an embodiment of the present invention;

FIG. 7 is a schematic diagram of the detection of cerebrospinal fluid and blood samples according to an embodiment of the present invention;

FIG. 8 is a schematic diagram of the stability of the kit according to an embodiment of the present invention;

FIG. 9 is a schematic diagram of the standard curve of chemiluminescence according to an embodiment of the present invention;

FIG. 10 is an electrophoresis identification diagram of antibody preparation and purification according to an embodiment of the present invention;

FIG. 11 is a schematic structural diagram of immunofluorescence according to an embodiment of the present invention; and

FIG. 12 is a schematic diagram of immunohistochemistry according to an embodiment of the present invention.

### DETAILED DESCRIPTION OF THE INVENTION

The embodiments of the present invention provide a monoclonal antibody against human AB and uses thereof, and those skilled in the art may refer to the content herein and appropriately improve the process parameters for implementation. It should be particularly noted all similar substitutions and changes are obvious to those skilled in the art and are deemed to be included in the invention. The method and uses of the present invention have been described by means of preferred embodiments. Obviously, relevant persons can implement and apply the present invention by making changes or appropriate variations and combinations to the method and uses herein without departing from the content, spirit and scope of the present invention.

In the embodiments of the present invention, a monoclonal antibody against Aβ42 is derived by a large number of screenings, the monoclonal antibody can bind to Aβ42 monomers in humans, mouses and monkeys and human Aβ42 aggregates, and has a relatively high affinity to the human Aβ42 aggregates, and it reduces the deposition of the Aβ42 aggregates by inhibiting and eliminating two mechanisms, and alleviates and slows down the neurotoxic effect resulting from the abnormal metabolism of Aβ42. Specifically, firstly, in a specific embodiment of the present invention, a monoclonal antibody AB7G, or a fragment thereof, against Aβ42 is provided; the monoclonal antibody AB7G or the fragment thereof is able to specifically bind to an Aβ42 monomer and an Aβ42 aggregate, in particular an Aβ42 oligomer; and the monoclonal antibody AB7G or the fragment thereof comprises a heavy chain variable region and a light chain variable region.

The sequence of the heavy chain variable region of the monoclonal antibody AB7G or the fragment thereof is: a sequence as set forth in SEQ ID NO: 2, or an amino acid sequence having the same functional protein and derived by deletion, substitution or insertion of one or more amino acids of the sequence as set forth in SEQ ID NO: 2. The sequence of the light chain variable region of the monoclonal antibody AB7G or the fragment thereof is: a sequence as set forth in SEQ ID NO: 4, or an amino acid sequence having the same functional protein and derived by deletion, substitution or insertion of one or more amino acids of the sequence as set forth in SEQ ID NO: 4.

A CDR region of the monoclonal antibody AB7G largely determines the affinity and specificity of an antibody-binding antigen, and the CDR region with the above sequences is conducive to improving the binding affinity and specificity of the monoclonal antibody AB7G to the Aβ42.

In an embodiment of the present invention, provided is a monoclonal antibody AB11A2 or a fragment thereof for quantitative detection of an Aβ42 monomer or an aggregate thereof in human body fluids in combination with the monoclonal antibody AB7G or the fragment thereof. The sequence of a heavy chain variable region of the monoclonal antibody AB11A2 or the fragment thereof is: a sequence as set forth in SEQ ID NO: 6, or an amino acid sequence having the same functional protein and derived by deletion, substitution or insertion of one or more amino acids of the sequence as set forth in SEQ ID NO: 6. The sequence of a light chain variable region of the monoclonal antibody AB11A2 or the fragment thereof is: a sequence as set forth in SEQ ID NO: 8, or an amino acid sequence having the same functional protein and derived by deletion, substitution or insertion of one or more amino acids of the sequence as set forth in SEQ ID NO: 8.

In the embodiment of the present invention, the “amino acid sequence having the same functional protein and derived by deletion, substitution or insertion of one or more amino acids” described above refers to a sequence that is different from the sequence as set forth at one or more amino acid residues but retains the biological activity of a resulting molecule, and it may be a “conservatively modified variant” or modified by “conservative amino acid substitution”. The “conservatively modified variant” or “conservative amino acid substitution” refers to an amino acid substitution known to those skilled in the art, and such a substitution generally does not alter the biological activity of the resulting molecule. In general, it is generally accepted by those skilled in the art that the substitution of a single amino acid in the functionally non-essential region of a monoclonal antibody substantially does not alter the biological activity.

In the present invention, “more” in the “amino acid sequence having the same functional protein and derived by deletion, substitution or insertion of one or more amino acids” is ≥2 and ≤30.

The monoclonal antibody AB7G, or the fragment thereof, against Aβ42 comprises any combination of the light chain variable region and the heavy chain variable region as follows: a sequence of the heavy chain variable region is as set forth in SEQ ID NO. 2 or is a sequence having at least 90% homology with the sequence set forth in SEQ ID NO. 2, and a sequence of the light chain variable region is as set forth in SEQ ID NO. 4 or is a sequence having at least 90% homology with the sequence as set forth in SEQ ID NO. 4; preferably, the homology is at least 95%; and more preferably, the homology is at least 98%.

The monoclonal antibody AB11A2 or the fragment thereof comprises any combination of the light chain variable region and the heavy chain variable region as follows: a sequence of the heavy chain variable region is as set forth in SEQ ID NO. 6 or is a sequence having at least 90% homology with the sequence set forth in SEQ ID NO. 6, and a sequence of the light chain variable region is as set forth in SEQ ID NO. 8 or is a sequence having at least 90% homology with the sequence as set forth in SEQ ID NO. 8; preferably, the homology is at least 95%; and more preferably, the homology is at least 98%.

The sequence with at least 90% homology or at least 95% or 98% homology means that its homology resulting from the homology alignment with the sequence of the antibody is not less than 90%, 95% or 98%, and it has the same function as the antibody, i.e., capability to bind to the Aβ42 monomer and the Aβ42 aggregate.

Based on the light chain and heavy chain variable regions of the above monoclonal antibody AB7G or the fragment thereof or of the monoclonal antibody AB11A2 or the fragment thereof, those skilled in the art can construct full-length monoclonal antibody molecules by conventional technical means in the art as needed.

According to the amino acid sequences of the heavy and light chains, those skilled in the art can design different nucleotide sequences capable of encoding the heavy and light chains according to the codon preference of an expression host, and all nucleic acids capable of encoding the light and heavy chains provided by the present invention should fall within the protection scope of the present invention.

In this embodiment, the nucleic acids encoding the heavy and light chain variable regions of the monoclonal antibody AB7G, or the fragment thereof, against Aβ42 have nucleotide sequences as follows: (1) a sequence of the nucleic acid encoding the heavy chain variable region is as set forth in SEQ ID NO. 1 or is a complementary sequence thereof; and a sequence of the nucleic acid encoding the light chain variable region is as set forth in SEQ ID NO. 3 or is a complementary sequence thereof; (2) a sequence that encodes the same monoclonal antibody or a fragment thereof as the nucleotide sequence of (1) but is different from the nucleotide sequence of (1) due to the degeneracy of the genetic code; (3) a sequence that has at least 80% homology with the sequence of (1) or (2); and (4) a nucleotide sequence that has the same function as the nucleotide sequence of (1), (2), or

(3) and is derived by substitution, deletion or addition of one or more nucleotides in the nucleotide sequence of (1), (2), or (3).

In this embodiment, the nucleic acids encoding the heavy and light chain variable regions of the monoclonal antibody AB11A2 or the fragment thereof have nucleotide sequences as follows: (5) a sequence of the nucleic acid encoding the heavy chain variable region is as set forth in SEQ ID NO. 5 or is a complementary sequence thereof; and a sequence of the nucleic acid encoding the light chain variable region is as set forth in SEQ ID NO. 7 or is a complementary sequence thereof; (6) a sequence that encodes the same monoclonal antibody or a fragment thereof as the nucleotide sequence of (5) but is different from the nucleotide sequence of (5) due to the degeneracy of the genetic code; (7) a sequence that has at least 80% homology with the sequence in (5) or (6); and (8) a nucleotide sequence that has the same function as the nucleotide sequence of (5), (6), or (7) and is derived by substitution, deletion or addition of one or more nucleotides in the nucleotide sequence of (1), (2), or (3).

In the embodiment of the present invention, “more” in “substitution, deletion or addition of one or more nucleotides” is ≥2 and ≤30.

In an embodiment of the present invention, further provided is a biological material comprising the nucleic acid described above, and the biological material is an expression cassette, a vector, a transposon, a host cell, an engineered bacterium or a genetically modified cell line.

The vector includes but is not limited to a cloning vector, an expression vector, and a plasmid vector, and all vectors comprising the nucleic acid encoding the monoclonal antibody AB7G, or the fragment thereof, against the Aβ42 monomer or aggregate or the monoclonal antibody AB11A2, or the fragment thereof, for detection of the Aβ42 monomers or aggregates should fall within the protection scope of the present invention.

The host cell or genetically modified cell line may be a cell or cell line derived from a microorganism, a plant or an animal, and all host cells or genetically modified cell lines of the vectors comprising the nucleic acid encoding the monoclonal antibody AB7G or the fragment thereof or the monoclonal antibody AB11A2 or the fragment thereof should fall within the protection scope of the present invention.

In an embodiment of the present invention, further provided is a complex or conjugate, which comprises the monoclonal antibody AB7G, or the fragment thereof, against the Aβ42 monomers or the aggregates thereof, or is prepared from the monoclonal antibody AB7G or the fragment thereof. In an embodiment of the present invention, further provided is a complex or conjugate, which comprises the monoclonal antibody AB11A2, or the fragment thereof, for detection of the Aβ42 monomers or the aggregates thereof, or is prepared from the monoclonal antibody AB11A2 or the fragments thereof.

The complex is derived by chemically or biologically labeling the monoclonal antibody AB7G, or the fragment thereof, against the Aβ42 monomers or aggregates. Or, the complex is derived by chemically or biologically labeling the monoclonal antibody AB11A2, or the fragment thereof, for detection of the Aβ42 monomer or aggregate. The chemically labeling involves but is not limited to isotopes, immunotoxins, and/or chemical drugs. The biologically labeling involves but is not limited to biotin, avidin or enzyme labels.

The conjugate is derived by conjugating the monoclonal antibody AB7G, or the fragment thereof, of the Aβ42 monomer or the aggregate thereof, or the chemically or biologically labeled complex, to a solid or semi-solid medium. Or, the conjugate is derived by conjugating the monoclonal antibody AB11A2, or the fragment thereof, for detection of the Aβ42 monomers or the aggregates thereof, or the chemically or biologically labeled complex, to a solid or semi-solid medium. The solid medium or non-solid medium includes but is not limited to colloidal gold, polystyrene flat plates or beads.

In an embodiment of the present invention, provided is use of the monoclonal antibody AB7G or the fragment thereof, or the nucleic acid encoding the monoclonal antibody AB7G or the fragment thereof, or the monoclonal antibody AB11A2 or the fragment thereof, or the nucleic acid encoding the monoclonal antibody AB11A2 or the fragment thereof, or the biological material, or the complex or conjugate, as defined above, for any one of: (1) preparation of a reagent and medicament for prevention, diagnosis or treatment of an Aβ42-driven amyloidosis; (2) preparation of a product for mitigation of polymeric toxicity of the Aβ42 or for control of development of the Aβ42-driven amyloidosis; (3) preparation of a product for inhibition of formation of the Aβ42 monomers or aggregates, fibrils, amyloid fibers or plaques; (4) preparation of a product for reduction of accumulation of the Aβ42 aggregates; and (5) preparation of a reagent or kit for detection of the Aβ42 monomers, the Aβ42 aggregates, and an Aβ42 antibody.

The Aβ42-driven amyloidosis in the present invention is a disease manifested as a result of changed Aβ42 metabolism, and comprises AD or complicated AD, etc.

In an embodiment of the present invention, provided is a medicament, which comprises or is prepared from the monoclonal antibody AB7G or the fragment thereof, or further comprises the monoclonal antibody AB7G or the fragment thereof. The medicament is for use in any one of: (1) prevention, diagnosis or treatment of an Aβ42-driven amyloidosis; (2) mitigation of polymeric toxicity of the Aβ42 or control of development of the Aβ42-driven amyloidosis; (3) inhibition of formation of the Aβ42 monomers or an aggregates, fibril or corpus fibrosum of the Aβ42; and (4) reduction of accumulation of the aggregates, fibrils or amyloid fibers.

The medicament is for use in the prevention, diagnosis or treatment of the Aβ42-driven amyloidosis, which comprises AD and complicated AD.

In an embodiment of the present invention, further provided is a kit for specific quantitative detection of an Aβ42 oligomer in human body fluids, comprising: the monoclonal antibody AB7G or the fragment thereof for capturing the Aβ42 monomer or the aggregate thereof; and the monoclonal antibody AB11A2 or the fragment thereof for detecting the Aβ42 monomer or the aggregate thereof. The monoclonal antibody AB7G or the fragment thereof is immobilized on a microplate, and the monoclonal antibody A B 11A2 or the fragment thereof is labeled with biotin.

In a specific embodiment of the present invention, provided is a kit for quantitative detection of an Aβ42 oligomer in human body fluids, and the kit comprises a variety of monoclonal antibodies specifically recognizing the Aβ42 oligomer, as well as a matching method and usage procedures thereof, and is for use in preparation of a diagnostic reagent for AD.

In the specific embodiment of the present invention, the monoclonal antibodies secreted by cultured hybridoma cells of the monoclonal antibodies AB7G and AB11A2 are used to prepare a microplate for detection purposes and a labeled detection antibody, and after screening and optimization, they are assembled into a kit for quantitative detection of the Aβ42 oligomers in human body fluids in a specific way. The resulting kit for quantitative detection of the Aβ42 oligomers, the components thereof, and the assembling method and usage procedures thereof can specifically and quantitatively detect the level of the Aβ42 oligomers in human body fluids such as cerebrospinal fluid or blood.

Based on the above assembled kit for detection of the Aβ42 oligomers, the components thereof, and the assembling method and usage procedures thereof, molecular modification, chips or microfluidics or other methods are used for construction and recombination for adaptation to chemiluminescence, single molecules, electrochemiluminescence or artificial intelligence processing systems, in order for use in early screening, early diagnosis and disease detection of AD.

The above kit can be used for quantitative detection of the amyloid oligomers in human body fluids, with AB7G as the capture antibody and biotinylated AB11A2 as the detection antibody. A calibration material for the kit is a set of amyloid oligomers at concentrations of 0 μg/mL, 3.9 pg/mL, 7.8 pg/mL, 15.6 pg/mL, 31.25 pg/mL, 62.5 pg/mL, and 125 μg/mL, respectively. The linear detection range of the kit is 3.9-125 pg/mL, with the lowest detection limit of 7.8 pg/mL. The kit specifically recognizes the Aβ42 oligomer and has no cross-reactivity with Aβ40 and tau proteins.

The capture antibody of the kit is a monoclonal antibody sourced from the cultured hybridoma cells AB7G, and the detection antibody of the kit is a monoclonal antibody sourced from cultured hybridoma cells AB11A2. The hybridoma cells AB7G and AB11A2 are both collected in the China Center for Type Culture Collection (CCTCC), with Accession Numbers of CCTCC C201256 and CCTCC C2012130 respectively. The heavy and light chain variable region genes of the monoclonal antibodies AB7G and AB11A2 can be recombined to express antibody active fragments that specifically recognize and bind to A B.

The monoclonal antibody AB7G may specifically recognize Aβ42 oligomers with an apparent molecular weight of 17-95 kDa and Aβ42 oligomers in the brain tissue of APP/PS1 genetically modified mice. Its subclass is IgG₃, the antigen recognition site is the C-terminal amino acid of the Aβ polypeptide, and the optimal dilutions for ELISA, Western blot, immunofluorescence and immunohistochemistry are 1:106, 1:1,000, 1:50 and 1:100.

In this embodiment, the light and heavy chain variable region genes are cloned to the monoclonal antibodies AB7G and AB11A2 by PCR. The heavy and light chain variable region genes are cloned from hybridoma cells capable of secreting highly active murine anti-Aβ42 monoclonal antibodies. Here, the heavy chain variable region gene of the monoclonal antibody AB7G is 474 bp, the heavy chain variable region gene of the monoclonal antibody AB11A2 is 447 bp, the light chain variable region gene of the monoclonal antibody AB7G is 384 bp, the light chain variable region gene of the monoclonal antibody AB11A2 is 348 bp, and the variable region genes may be recombined to express an antibody active fragment that specifically recognizes and binds to the Aβ42 oligomers.

Here, the DNA residue sequence of the heavy chain variable region of the monoclonal antibody AB7G is as set forth in SEQ ID NO: 1; the amino acid residue sequence of the heavy chain variable region of the monoclonal antibody AB7G is as set forth in SEQ ID NO: 2; the DNA residue sequence of the light chain variable region of the monoclonal antibody AB7G is as set forth in SEQ ID NO: 3; the amino acid residue sequence of the light chain variable region of the monoclonal antibody AB7G is as set forth in SEQ ID NO: 4; the DNA residue sequence of the heavy chain variable region of the monoclonal antibody AB11A2 is as set forth in SEQ ID NO: 5; the amino acid residue sequence of the heavy chain variable region of the monoclonal antibody AB11A2 is as set forth in SEQ ID NO: 6; the DNA residue sequence of the light chain variable region of the monoclonal antibody AB11A2 is as set forth in SEQ ID NO: 7; and the amino acid residue sequence of the light chain variable region of the monoclonal antibody AB11A2 is as set forth in SEQ ID NO: 8.

The cell strains secreting the above-mentioned monoclonal antibodies against AD are two murine anti-human Aβ42 monoclonal antibody hybridoma cell lines, named AB7G and AB11A2, obtained by traditional fusion of myeloma and splenocytes. The hybridoma cells AB7G and AB11A2 have been collected in the China Center for Type Culture Collection (CCTCC). See the biological collection information for details.

Combined with the above resulting composition of capture and detection antibodies in the kit for quantitative detection of amyloid in human body fluids in this embodiment, and in conjunction with the enzyme labeling, luminescent material labeling, magnetic particle binding, chip, colloidal gold, microfluidics and other techniques, reagents for detecting AD for use in at least 6 different methods such as double-antibody sandwich ELISA (enzyme-linked immunosorbent assay), plate chemiluminescence, tubular magnetic particle luminescence, electrochemiluminescence, test card and liquid chips may be prepared respectively.

Based on the above 6 reagents, a combination of specific devices may be used to detect the amyloid in body fluids such as cerebrospinal fluid, blood, urine, or saliva related to the AD.

With the above anti-human Aβ monoclonal antibody alone, reagents for detecting AD for use in at least 4 different methods such as ELISA (enzyme-linked immunosorbent assay), Western blot assay, immunofluorescence and immunohistochemistry may be prepared respectively.

Based on the above-mentioned light and heavy chain variable region genes cloned to the monoclonal antibody against the human Aβ42 oligomer, genetic engineering may be used to construct and/or express a variety of vectors and cell strains of small molecule genetically engineered antibodies, and antibodies such as single-chain antibodies, chimeric antibodies and Fab antibodies, in order for use in basic and clinical research and application for the diagnosis, prevention and treatment of AD. Therefore, the monoclonal antibody against human Aβ42 may also be prepared into a medicament for the diagnosis, prevention and treatment of the AD. The gene nucleic acid sequence is cloned to a specific expression vector to transfect/transform corresponding recipient cells (including bacteria, mammalian cells and yeast, etc.), to obtain a cell strain with high expression of the antibody molecule. In this embodiment, the following experiments are carried out in the preparation of the kit and the identification of the monoclonal antibody against human Aβ42 and its properties and functions.
1. Preparation of kit and establishment of testing procedures and calibration curves, including the coating of a microplate, the preparation of the detection antibody and a detection method, the determination of a linear range, the lowest detection limit, and the sensitivity. 2. Preparation, optimization and identification of calibration material, including the preparation and identification of an Aβ42 oligomer mixture. 3. Preparation of immunogen and antibody and identification of antibody properties. The soluble Aβ42 oligomers are the leading early pathogenic factor of AD and show the highest toxicity to neurons. Currently, it relates to the monoclonal antibody targeting the Aβ42 oligomer of 17-95 kD for diagnostic and therapeutic purposes. 4. Indirect ELISA experiment. The results show that AB7G may well recognize the Aβ oligomer mixture coated in a 96-well microplate with an alkaline coating solution. The lowest detection limit is 5 ng/ml. The optimal dilution for ELISA is 1:106.5. Western blot experiment. The results indicate: AB7G may well recognize the AB oligomer mixture (mainly the oligomers of 17-95 kD) that is separated by SDS-PAGE and transferred to a nitrocellulose membrane. The optimal dilution for Western blot is 1:4,000.6. Antibody matching and screening experiment by double-antibody sandwich ELISA. After experiments and screening of various matching methods, AB7G and AB11A2 are determined to be the optimal antibody matching method in the dual-antibody sandwich ELISA. The content of the coated antibody is selected to be 8 μ/well. 7. Optimization of antibody dilution by checkerboard titration experiment. 8. Specificity of detection. 9. Operation methods and sample testing. 10. Spike recovery, stability and precision. 11. Other uses of calibration curves. Different detection systems are adapted to chemiluminescence and different models of devices. 12. Comparison of multiple kits. The performances of several similar kits are compared. 13. Immunofluorescence experiment. 14. Immunohistochemistry experiment. The monoclonal antibody AB7G may specifically recognize Aβ oligomers in brain tissues of senescence accelerated mouse P8 (SAM P8). The results show that clear staining and accurate signal locating. 15. Cloning experiment of variable region genes of monoclonal antibodies. The light and heavy chain variable region genes of anti-monoclonal antibodies are amplified by 5′RACE and RT-PCR. 16. Cell protection experiment. The cells treated with the monoclonal antibody AB7G are able to counteract the cytotoxicity of Aβ oligomers.

### Experiment 1: Procedures of Kit Detection and Generation of Calibration Curves

### 1. Methodology

### 1.1 Preparation of Antibody-Coated Microplate

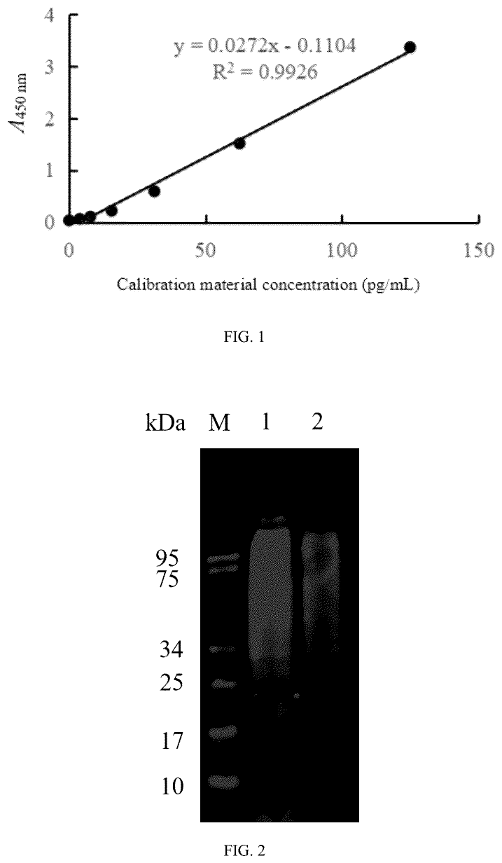

Coating: the capture antibody (AB7G) was diluted with PBS (containing 0.05% biopreservative) and added to a single strip of removable 96-well microplate at 50 μL/well (8 μg), and stayed at 4° C. overnight.
Blocking: The microplate was washed three times with 1×PBST, each for 3 min. The microplate was then blocked with 10% FBS at 200 μL/well, incubated at 37° C. for 2 h, and washed 3 times with 1×PBST, each for 3 min.
   1.2 Dilution of calibration material. The calibration material was diluted with 10% FBS to 0 pg/mL, 3.9 pg/mL, 7.8 pg/mL, 15.6 pg/mL, 31.2 pg/mL, 62.5 pg/mL, and 125 μg/mL, with 50 μL added per well.
   1.3 Preparation of detection antibody and streptavidin-HRP Solution. Detection antibody: the AB11A2-Bio antibody was diluted at 1:2000 with 10% FBS, followed by the addition of 0.05% biopreservative. Streptavidin-HRP: Streptavidin-HRP was diluted at 1:2,000 with 10% FBS, followed by the addition of 0.05% biopreservative.
   1.4 Addition of sample: Calibration wells, negative wells, blank wells, and test sample wells were set up. 50 μL of 10% FBS was added to the negative wells, and 50 μL of a calibration material, 12.5 μL of a test sample and 37.5 μL of a diluent were added to the calibration wells and the test sample wells, respectively. 50 μL of the detection antibody was added per well except for blank wells. After gently shaking and evenly mixing, the wells were covered with a membrane and incubated at room temperature for 1-2 h, the liquid in the wells was discarded, the wells were spun dry, and the microplate was washed 5 times with 1× PB ST.
   1.5 Except for the blank wells, 100 μL of a streptavidin-HRP solution (BioLegend) was added to each of the standard wells and the test sample wells, and after incubation at room temperature for 0.5 h, the liquid in the wells was discarded, the wells were spun dry, and the microplate was washed 5 times with 1×PBST.
   1.6 Addition of chromogenic substrate: 100 μL of a TMB substrate (BioFx® TMB Super Sensitive One Component HRP Microwell Substrate) was added to each well, and incubated at room temperature in the dark for 10 min, and the incubation was terminated when a significant blue gradient appeared.
   1.7 Stop: 50 μL of a stop solution (0.5 M H₂SO₄) was added per well. The reaction was terminated, at which point the blue color immediately turned to yellow.
   1.8 Reading: the OD value of each well (A450) was measured with a microplate reader at a wavelength of 450 nm.
   1.9 Drawing of calibration curve: the OD value of the blank well was subtracted from the OD value of each calibration material for plotting, and the average value of multiple wells was calculated. A calibration curve was plotted by taking the concentration of the calibration material as the abscissa and the OD value as the ordinate.
   2. Results The calibration curve was shown in FIG. 1, with the linear range of 3.9-125 pg/mL, the lowest detection limit of 7.8 pg/mL, and the R 2 value of above 0.99. It indicated that the composition kit of the present invention had a reasonable linear detection range and high sensitivity.

### Experiment 2: Preparation of Calibration Material Aβ42 Oligomer

The synthesis of Aβ42 peptides was entrusted to Shanghai Sangon Bioengineering Technology Service Co., Ltd. Referring to the method in literature (Lambert M, 1998) and making necessary adaptation, an Aβ42 oligomer mixture was obtained by carrying out in vitro assembly under near-native conditions. First, 1 mg of the Aβ42 peptide was dissolved in ice-chilled hexafluoroisopropanol (1,1,1,3,3,3-hexafluoro-2-propanol, HFIP) (Sigma) to monomerize the Aβ42 peptide, and stayed at room temperature for 1 h to volatilize the HFIP completely. Then, the AB1-42 monomer was dissolved with 20 μL of anhydrous dimethyl sulfoxide (DMSO) (Sigma), finally placed in an F12 medium (Sigma) or a phosphate buffer system (with the volume supplemented to 1 mL accordingly), and stayed at 4° C. for 24 h for natural polymerization, and the prepared Aβ42 oligomer mixture was detected by Western blot. See FIG. 2.

### Experiment 3: Preparation of Capture and Detection Antibodies

1. Design and Synthesis of Immunogen. The immunogen was composed of peptide fragments KLH-GGVVIA (SEQ ID NO: 9) and KLH-LVFFAEDV (SEQ ID NO: 10), and was synthesized by Shanghai Sangon Bioengineering Co., Ltd.
2. Immunity in Mice. Three female BALB/c mice aged 6-8 weeks were immunized 4 times as follows: (1) the immunogen at a dose of 50 μg/mouse was mixed with the same volume of Freund's complete adjuvant (Sigma) and fully emulsified for intraperitoneal injection; (2) 2 weeks later, the immunogen at a dose of 30 μg/mouse was mixed with the same volume of Freund's complete adjuvant (Sigma) and fully emulsified for intraperitoneal injection; (3) 2 weeks later, (2) was repeated; and (4) two weeks later, rush immunization was carried out by intraperitoneal injection with 50 μg of immunogen, and 3-4 days later, the spleen was harvested for fusion.
3. Preparation of feeder cells: BALB/c mice aged 6-8 weeks were sacrificed by pulling the neck, an RPMI 1640 culture solution (Sigma) was used to flush the peritoneal cavity several times under sterile conditions, and the flushing solution was centrifuged at 2,000 rpm for 10 min. The supernatant was discarded, pellets were suspended with an RPMI 1640 culture solution containing 20% fetal bovine serum, the cells were counted, adjusted in concentration to 10⁵ cells/mL, added to a 96-well microplate at 100 μL/well, and placed in a 5% CO₂ incubator at 37° C.
4. Cell fusion: The mice rush-immunized for 3-4 days were subjected to eye bleeding to collect serum, and then sacrificed to remove spleens aseptically for preparation of a single-cell suspension; splenocytes were mixed with Sp2/0 cells in the logarithmic growth phase (screened by 8-nitroguanine, i.e., 8-azaguanine, referred to as 8-AG) at a ratio of 4:1, centrifuged at 1,000 rpm for 10 min, and the supernatant was discarded; the centrifuge tube was flicked at the bottom to turn pellet cells into paste, and placed into a water bath at 37° C., followed by the addition of 1 mL of 50% PEG 1500 (polyethylene glycol, Sigma) within 1 min over rotating to keep the cells in an evenly mixed state; the tube stood for 90 s in the water bath at 37° C.; 20 mL of an RPMI 1640 solution was immediately added slowly; the tube was centrifuged at 800 rpm for 8 min, and the supernatant was discarded; a −RPMI 1640 solution containing 20% fetal bovine serum (Hangzhou Sijiqing Bioengineering Materials Co., Ltd.), 2% HAT (Sigma), and 1% penicillin (Sigma) was added; the mixture was gently mixed well, and the cell concentration was adjusted to 2×10⁶ cells/mL; the resulting mixture was added to the 96-well cell culture microplate with feeder cells at 100 μL/well, and placed in a 5% CO₂ incubator at 37° C.; cell growth was observed day by day; after 7-10 days, the RPMI 1640 culture solution containing 20% calf serum, 1% HT (Sigma) and 1% penicillin was added; and the clone growth rate was calculated according to the following formula. Clone growth rate (%)=(cell clone growth wells/inoculation wells)×100%.
5. Screening and Subcloning of Positive Clones. When the cell clones grew to ⅓ of the field of view (microscope magnification 4×10), the cell supernatant was carefully pipetted, 100 ng/mL Aβ oligomer mixture was the coating antigen, and the antibodies in the supernatant were detected by indirect ELISA. See “4. Cell fusion” for the specific method and judgment criteria. The clone positive rate was calculated according to the following formula: Clone positive rate (%)=(antibody positive wells/cell clone growth wells detected)×100%. The wells with positive antibody secretion were cloned repeatedly by limiting dilution until the antibody positive rate of the supernatant in all monoclonal wells was 100%.
6. Establishment of Hybridoma Cell Strains. The cloned positive clones were transferred into a 24-well microplate, a 6-well microplate, and a T25 cell culture flask, and cultured in an expanded way for more than 90 days, and the cell strains were preserved.
7. Subclass Identification of Monoclonal Antibodies. According to the operation procedures of the mouse IgG subclass identification kit (Sigma), the IgG subclass of the monoclonal antibody AB7G secreted by the hybridoma cell strain was IgG₃. The IgG subclass of the monoclonal antibody AB11A2 secreted by the hybridoma cell strain was IgG2a.
8. Epitope Analysis of Monoclonal Antibodies. Antigen-recognition epitopes of monoclonal antibodies were determined by the competitive ELISA as follows: the monoclonal antibody was incubated with a short peptide fragment in a concentration gradient for 1 h, and then added to a microplate coated with an Aβ42 oligomer for indirect ELISA detection. The results showed that the AB7G epitope was C-terminal GVVIA.
9. Massive Preparation and Purification of Monoclonal Antibodies. The hybridoma cells were inoculated into the peritoneal cavity of a BABL/c mice pretreated with norphytane (Sigma), 1×10⁷ hybridoma cells/mouse; ascites was extracted 7-10 days later; the monoclonal antibodies were purified by Protein A affinity chromatography columns using an AKTA explore 100 protein chromatography system; and the heavy and light chains of the antibodies were visible by SDS-PAGE Coomassie brilliant blue staining. See lanes 2 and 3 in FIG. 10. The antibody concentration was determined with a BCA kit (PIERCE, USA) at 4 mg/mL.

In addition, the mice were immunized with several antigens such as Aβ1-12-KLH and KLH-AB30-42, and a plurality of monoclonal antibody cell strains such as ABW and ABC13 were screened for subsequent screening experiments.

### Experiment 4: Indirect ELISA Experiment

1. Method: (1) Coating: the 200 ng/mL Aβ42 oligomer was added to a microplate (Corning) at 100 μL per well and stayed at 4° C. overnight. At the same time, a control group coated with Aβ42 unpolymerized peptides (200 ng/ml) was set up. (2) Microplate washing three times with PBS-T (8 g of NaCl, 0.2 g of K CI, 1.44 g of Na₂HPO₄, 0.44 g of KH₂PO₄, and 0.05 mL of Tween-20, added with deionized water to 1 L, and with pH adjusted to 7.2-7.4), each for 5 min. (3) Blocking: 100 μL of PBS-T containing 0.2% BSA was added to each well and incubated at 37° C. for 2 h. (4) The double-diluted monoclonal antibody was added to a 96-well microplate at 100 μL per well. The microplate was incubated at 37° C. for 2 h. At the same time, blank control wells, negative control wells and positive control wells were set up. (5) Microplate washing with PBS-T: 3 times, each for 5 min. (6) Goat anti-mouse IgG-HRP (Beijing Zhongshan Golden Bridge Biological Technology CO., LTD.) diluted at 1:10,000 was added at 100 μL per well, at 37° C. for 1 h. (7) Microplate washing with PBS-T: 3 times, each for 5 min. (8) A substrate TMB was added at 100 μL per well, and after 20 min, the OD value at the wavelength of 450 nm was measured.

The criteria for judging positivity were as follows: (OD value of sample well-OD value of blank control)/(OD value of negative control well-OD value of blank control) ≥2. The maximum dilution of the monoclonal antibody was the titer of the antibody.
2. Results and Conclusions: The prepared monoclonal antibodies AB7G and AB11A2 were able to recognize the coated Aβ42 oligomers (average OD values up to 3.319 and 3.205, respectively), but showed low recognition ability for unpolymerized Aβ peptides (average OD value of 0.065). The titer of mouse ascites prepared from the monoclonal antibody reached over 1:2,000,000, and the titer of the purified antibody reached over 1:512,000, with the lowest detection limit of 5 ng/ml.

The above results showed that the monoclonal antibodies AB7G and AB11A2 could effectively recognize the Aβ oligomers assembled in vitro, and had good response sensitivity.

### Experiment 5: Western Blot Experiment

### 1. Methodology

0.5-1 μg of the sample and 5× of sample buffer were mixed well and boiled at 100° C. for 5 min, followed by the addition of a sample, and a 100 V voltage was first applied to allow the protein to pass through the stacking gel. When the sample entered the separation gel, the voltage was adjusted to keep constant at 120 V. When the bromophenol blue swam to the bottom of the gel, the electrophoresis was ended, the gel was removed, and staining was carried out with Coomassie brilliant blue R-250. The gel and the nitrocellulose membrane were placed into a container filled with a blotting buffer and equilibrated for 10 min; the filter paper, the gel, the NC membrane, and the filter paper were then placed in order to form a “sandwich” shape; a membrane transfer buffer was added, with a gel side facing the negative electrode, and the NC membrane side facing the positive electrode; and air bubbles should be carefully avoided and removed. The power supply was turned on, and the constant current of 80 mA was continuously transferred for 2 h.

After membrane transfer, the position of the protein band was determined with a Ponceau S staining solution (the preparation method of 10× Ponceau S stock solution was as follows: weighing 2 g of the Ponceau S, 30 g of trichloroacetic acid, and 30 g of sulfosalicylic acid, and adding water to 100 ml; and when in use, the resulting mixture was diluted with deionized water at a ratio of 1:10), and marked accordingly. The nitrocellulose membrane was blocked with a blocking solution (prepared by dissolving 5 g of skimmed milk powder in 0.1 mol/L PBST (8 g of NaCl, 0.2 g of KCl, 1.44 g of Na₂HPO₄, 0.44 g of KH₂PO₄, and 0.05 mL of Tween-20, added with deionized water to 1 L, and with pH adjusted to 7.2-7.4), and stayed at 4° C. overnight. The monoclonal antibodies were diluted with the blocking solution and incubated at 4° C. for 12-14 h. The nitrocellulose membrane was washed with 0.1 mol/L PBST 4 times, each for 5-10 min. The NIR dye-labeled antibodies (IRDye 680RD Donkey anti-Mouse Secondary Antibodies) (1:10,000) were diluted with the blocking solution and incubated for 1 h at room temperature. The nitrocellulose membrane was washed 4 times with 0.1 mol/L PBST, each for 5 min. The results of Western blot were scanned and analyzed by a near-infrared laser imaging system (LI-COR Odyssey CIx Imager).

2. Results and Conclusions: The Western blot results showed that the purified monoclonal antibody AB7G mainly recognized the Aβ42 oligomers in the range of 17-95 kD. The results showed that the monoclonal antibody AB7G had the maximum response to the oligomers of 17-95 kD in the AB oligomer mixture, and the monoclonal antibody AB7G could be used for the detection of the Aβ42 oligomers in samples by Western blot.

### Example 6 Antibody Matching and Screening

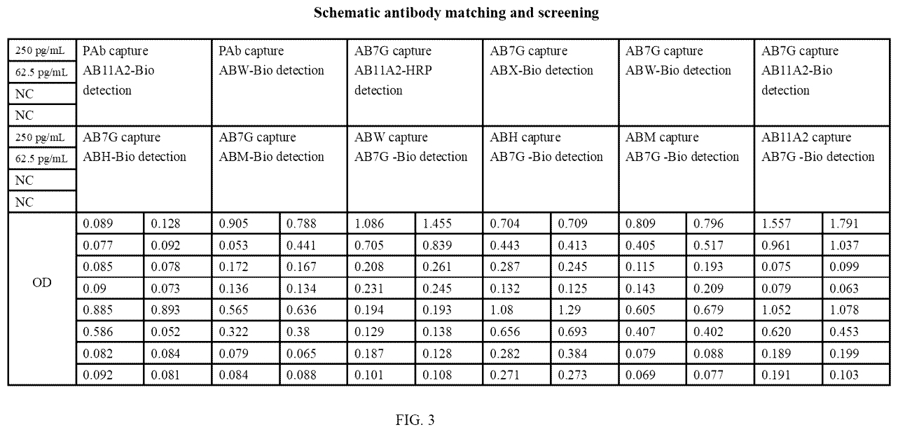

In order to screen for the optimal antibody matching method, the pairing experiments using a variety of antibodies were conducted, in which combination optimization was carried out among candidate antibodies PAb, AB7G, ABW, ABH, ABM, AB11A2, AB11A2-Bio, AB7G-Bio, ABM-Bio, ABW-Bio and ABX-Bio, and the calibration materials of 250 μg/mL and 62.5 pg/mL were detected by double-antibody sandwich ELISA, and the results showed that the matching combination of antibodies AB7G and AB11A2-Bio was the most effective in detection. See FIG. 3.

If AB7G and AB11A2-Bio were reversed in order, that is, AB11A2 was used to coat the microplate and AB7G was used as the detection antibody, the OD value of the positive signal decreased (P<0.05), and the OD value of the negative signal increased (P<0.05), resulting in a significant decrease in the P/N ratio (P<0.05), as shown in Table 1.

TABLE 1
Reduced Detection Effect after Reversing of AB7G and AB11A2

| Antibody matching | | | |
|---|---|---|---|
| (plate | | | |
| coating/detection) | Positive value | Negative value | P/N |
| AB11A2/AB7G | 1.069 ± 0.015 | 0.192 ± 0.001 | 5.56 ± 0.14 |
| AB7G/AB11A2 | 1.713 ± 0.135 | 0.091 ± 0.013 | 18.98 ± 1.54 |

At the same time, the monoclonal antibody ABW obtained by Aβ1-12 immunoscreening was combined with AB7G, or the matching order of the coated antibody and the detection antibody was reversed. Compared with the antibody combination method of the present invention (AB7G/AB11A2), the OD value of the positive signal decreased (P<0.05), and the OD value of the negative signal increased (P<0.05), resulting in a significant decrease in the P/N ratio (P<0.05), see Table 2.

TABLE 2
Reduced Detection Effect after Change
to Combination of ABW and AB7G

| Antibody matching | | | |
|---|---|---|---|
| (plate | | | |
| coating/detection) | Positive value | Negative value | P/N |
| ABW/AB7G | 0.193 ± 0.001 | 0.147 ± 0.034 | 1.351 ± 0.271 |
| AB7G/ABW | 0.800 ± 0.007 | 0.167 ± 0.045 | 5.094 ± 1.680 |

In addition, the monoclonal antibody ABC13 obtained by Aβ30-42 immunoscreening was combined with AB11A2 and reversed. Then, the OD value of the positive signal decreased (P<0.05), and the OD value of the negative signal increased (P<0.05), resulting in a significant decrease in the P/N ratio (P<0.05), as shown in Table 3.

TABLE 3
Reduced Detection Effect after Change
to Combination of ABC13 and AB11A2

| Antibody matching | | | |
|---|---|---|---|
| (plate | | | |
| coating/detection) | Positive value | Negative value | P/N |
| ABC13/AB11A2 | 0.503 ± 0.024 | 0.156 ± 0.027 | 3.301 ± 0.648 |
| AB11A2/ABC13 | 0.237 ± 0.013 | 0.194 ± 0.005 | 1.223 ± 0.096 |

Afterwards, a variety of other matching methods were used, and after comparison, it was found that the matching combination of antibodies AB7G and AB11A2-Bio had the best detection effect. On this basis, the optimization of the detection system was proceeded.

### Experiment 7: Optimization of Detection System

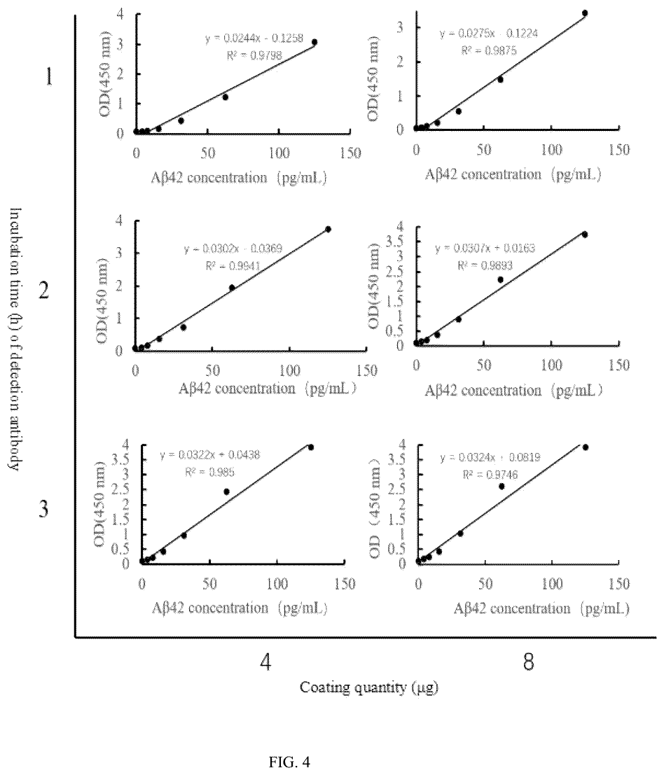

For the matching method taking AB7G as the capture antibody and AB11A2-Bio as the detection antibody, the reaction conditions of the double-antibody sandwich ELISA detection system were further optimized, including the comparison and adjustment of the reaction time, antibody concentration, dosage of microplate coating antibody, reaction temperature and other conditions of each step. The final reaction conditions were as follows: the monoclonal antibody AB7G was used to coat the microplate at 8 μg/well, the sample and AB11A2-Bio were added, the resulting mixture was incubated for 1-2 h at room temperature and allowed to react for 0.5 h at room temperature, and the AB11A2-Bio was diluted at 1:2,000. See FIG. 4.

### Experiment 8: Specificity of Detection System

Aβ42, AB40 and recombinant human tau protein (SIGMA) were prepared at 0 μg/mL, 3.9 pg/mL, 7.8 pg/mL, 15.6 pg/mL, 31.2 pg/mL, 62.5 pg/mL and 125 μg/mL, respectively, for detection with the kit of this embodiment. The results showed that this detection system had the specificity to Aβ42 and did not recognize the AB40 and the recombinant human tau protein. See FIG. 5.

### Experiment 9: Sample Testing

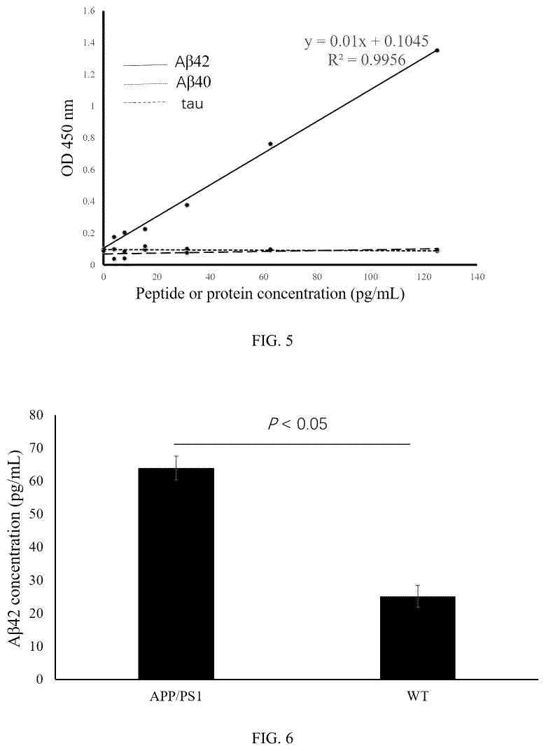

5 μL samples of brain tissue cell extracted from AD genetically modified mice APPswe/PS1_{ΔE9} (APP/PS1) and wild-type (WT) mice were detected with the kit of the present invention. The results showed that the content of Aβ42 in the brain tissues of the APP/PS1 mice was significantly higher than that of WT. It suggested that this detection system could be used for the quantitative detection of Aβ42 in brain tissue cell proteins of AD genetically modified mice. See FIG. 6.

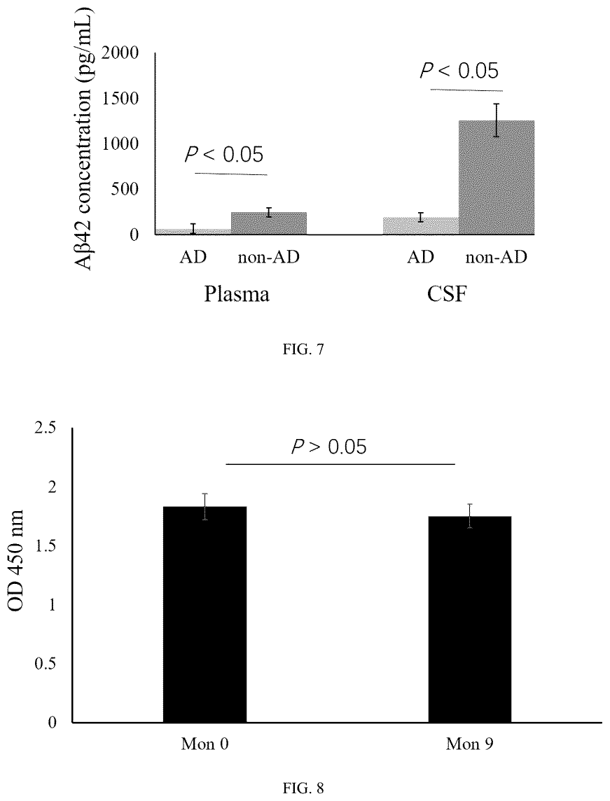

5 L of cerebrospinal fluid samples and 12.5 μL of plasma samples were taken from AD patients respectively, added to the sample wells and detected with the kit of the present invention. The results showed that the detection results of the AD group were significantly different from those of the non-AD group (P<0.05). It suggested that this kit could be used for the quantitative detection of amyloid in human body fluids. See FIG. 7.

### Experiment 10: Kit Stability Test

After preparation (Month 0), the kit was stored at 4° C. for 9 months (M on 9), and samples were taken 3 times to test the detection effect of the kit. The results showed that there was no significant difference between Mon 0 and M on 9 (P>0.05). It indicated that this kit could be stored at 4° C. for 9 months, with better stability. See FIG. 8. At the same time, samples were taken from the same batch for detection 3 times, and from three different batches for detection. The results showed that the CV value of the kit was less than 10%, indicating high precision of the kit.

### Experiment 11: Application of Matching Antibodies in Chemiluminescence System

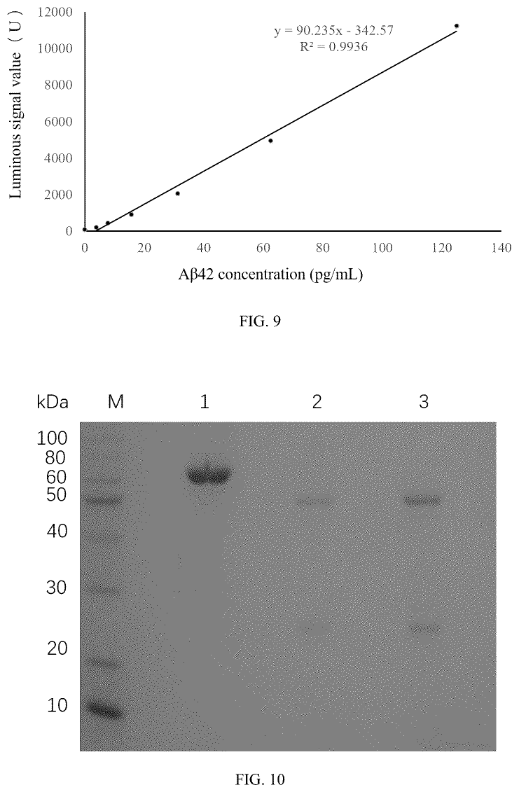

The same antibody matching was used with a chemiluminescent substrate, which also resulted in a comparable calibration curve. It suggested that the monoclonal antibody composition of the present invention was also suitable for the chemiluminescence detection system. See FIG. 9.

### Experiment 12: Comparison of Detection Performances of Kits from Different Sources

The kit was assembled using the antibodies prepared in this embodiment (FIG. 10). This kit was compared with similar products in China and abroad, and it could be seen that this kit had advantages in linear range, lowest detection limit, time consumption and sample size. See Table 4.

TABLE 4
Comparison of Parameters of Aβ42
Detection Kits from Different Sources

| | | Lowest | | | | | |
|---|---|---|---|---|---|---|---|
| | | detec- | | Time | | | |
| | Linear | tion | | consump- | | Sample | |
| List of kits | range | limit | Uses | tion | | size | |
| Foreign | 62.5-4,000 | 62.5 | Cerebro- | >2.5 | h | 25 | μL |
| product | pg/mL | pg/mL | spinal | | | | |
| (INNOTEST) | | | fluid | | | | |
| Domestic | 31.25-500 | 31.25 | Serum | 2.5 | h | 100 | μL |
| product | pg/mL | pg/mL | | | | | |
| (Anqun | | | | | | | |
| Biotech) | | | | | | | |
| Kit of the | 3.9-125 | 7.8 | Cerebro- | <2.5 | h | <12.5 | μL |
| present | pg/mL | pg/mL | spinal | | | | |
| invention | | | fluid and | | | | |
| | | | blood | | | | |

### Experiment 13: Immunofluorescence

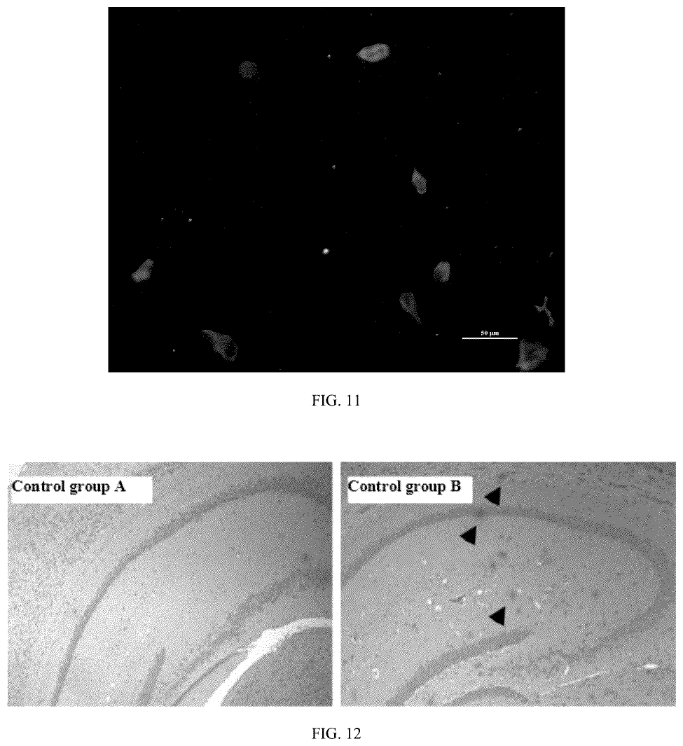

Neuroblastoma cells N2a were grown in a 96-well cell culture microplate, immobilized with paraformaldehyde, incubated with AB7G (1:50) overnight at 4° C., and incubated with fluorescently labeled goat anti-mouse IgG (1:200) at 37° C. for 1 h, and observation and photographing were carried out under a fluorescence microscope, see FIG. 11.

### Experiment 14: Immunohistochemistry Experiment

### 1. Methodology

First, paraffin sections were dewaxed, hydrated, and washed twice with PBS, each for 5 min; fresh 3% H₂O₂ was prepared with distilled water or PBS, blocked at room temperature for 5-10 min, and washed 3 times with distilled water; after antigen retrieval, washing was conducted with PBS for 5 min; a goat serum blocking solution was added dropwise and stayed at room temperature for 20 min, and the resulting mixture was shaken away to remove the excess liquid; the monoclonal antibody was added dropwise, stayed at room temperature for 1 h or at 4° C. overnight (rewarmed at 37° C. for 45 min after staying at 4° C. overnight), and was washed 3 times with PBS, each for 2 min; the biotinylated secondary antibody (goat anti-mouse IgG) was added dropwise, stayed at 20° C.-37° C. for 20 min, and was washed 3 times with PBS, each for 2 min; a reagent SA BC was added dropwise, stayed at 20° C.-37° C. for 20 min, was washed 4 times with PBS, each for 5 min; the mixture was subjected to color development with DAB, washed with distilled water, re-stained with hematoxylin for 2 min, and differentiated with hydrochloric alcohol; and the mixture was then subjected to dehydration, transparent treatment, mounting, and microscopic photographing.

### 2. Results and Conclusions

The results showed that the cerebral cortex and hippocampus were clearly stained, with accurate locating and low background. See FIG. 12. The monoclonal antibody AB7G could be used as an antibody for the detection of Aβ in the cerebral cortex and hippocampus.

### Experiment 15: Cloning of Variable Region Genes of Monoclonal Antibodies

The hybridoma cells AB7G and AB11A2 (5×10⁶) in the logarithmic growth phase were extracted by a Trizol reagent method to extract the total RNA of hybridoma cell strains, and a small amount of the total RNA was quantified by the ultraviolet spectrophotometer and subjected to 1% formaldehyde-denatured agarose gel electrophoresis. The light and heavy chain variable region genes of the monoclonal antibody against A β oligomers were amplified by 5′RACE and RT-PCR. Gene-specific primers CTGGATGGTGGGAAGATGG (SEQ ID NO: 11) and CAAGGGATAGACAGATGGGGC (SEQ ID NO: 12) were designed. The first strand of cDNA was synthesized using SuperScripII reverse transcriptase with the total RNA as a template according to the instructions of the kit. The reverse transcriptional reaction protocol was as follows: 5 μg of total RNA and 100 nmol/L GSP were supplemented with Rnase-free water to 18 μL, incubated at 70° C. for 10 min, and then placed on an ice bath immediately; 2.5 μL of 10× buffer, 2.5 μL of 25 mmol/L MgCl₂, and 1 μL of 10 mmol/L dNTP were then added to the reaction solution, and incubated at 42° C. for 1 min, followed by the addition of 1 μL of 200 U/μL SuperScripII. reverse transcriptase; and the resulting mixture was allowed to react at 42° C. for 50 min, and incubated at 70° C. for 10 min. After reverse transcription, RNase was added to hydrolyze the RNA, such that the product contained only the first strand of cDNA. The first strand of cDNA was purified using a S.N.A.P centrifuge column, and then a Poly (C) tail was added to the 5′-terminal of the first strand of cDNA with dCTP as a substrate by means of the terminal deoxynucleotidyl transferase TdT.

The light and heavy chain (VL, VH) variable region genes of the antibody AB7G were amplified with the first stand of cDNA, having the Poly (C) tail added, as a template by using two primer pairs, including
AAP:GGCCACGCGTCGACTAGTACGGGIIGGGIIGGGIIG (SEQ ID NO: 13) and L-GSP2 as well as AAP and H-GSP2, in the 5′RACE kit.

The light chain and heavy chain (VL, VH) variable region genes of the antibody AB11A2 were amplified by using ATGGATTTW(A,T)CAGGTGCAGATTW(A,T)TCAGCTTC (SEQ ID NO: 14) and CAGTGGATAGACCGATGGGGG (SEQ ID NO: 15), as well as ACTGGATGGTGGGAAGATGG (SEQ ID NO: 16) and ATGGAGW (A,T)CAGACACACTCCTGY(CT)TAY(C,T)GGGTG (SEQ ID NO: 17).

The VH and VL of the PCR amplification products were separated by agarose gel (1.5%) electrophoresis, and recovered and purified with a PCR product purification kit (Omega), and the target fragment was cloned into a T vector for sequencing analysis (see Sequence List 1-8).

The PCR amplification reaction was carried out according to the conventional method: the light and heavy chain variable region genes of the antibody were amplified by using the heavy and light chain primers, respectively, with the above product as a template. The reaction system was as follows: deionized water was added to 5 μL of cDNA, upstream and downstream primers (10 mmol/L, 1 μL for each), 2 μL of 10 mmol/L dNTP, 5 μL of 10× buffer, 1.25 μl of Ex Taq DNA polymerase to 50 μL, mixed well, instantaneously centrifuged, and then placed in the PCR instrument for reaction. The PCR conditions were: pre-denaturation at 95 for 5 min; cycling with the parameters of denaturation at 94 for 40 s, annealing at 55 for 40 s, and extension at 72 for 1 min, for a total of 30 cycles; and final extension at 72 for 51 min.

The cloning and sequencing protocol of the T vector of the target fragment was as follows: the PCR product was purified and recovered with a kit (Omega) and then ligated to a pMD18-T vector. The ligation reaction system was as follows: 1 μL of the pMD18-T vector, 4 μL of the purified heavy chain (or light chain) of the PCR product, and 5 μL of a ligation buffer were mixed well, then stayed at 4° C. overnight, and were transformed into E. coli DH5a, and the recombinant clones were screened and sequenced.

The light and heavy chain variable region genes of the present invention were reconstructed into a certain form of protein medicament, which could be directly used in the diagnosis and immunotherapy of AD.

### Experiment 16: Cell Protection Experiment

The cells in the logarithmic growth phase N2a were seeded into a 96-well microplate and divided into a cell control group, a monoclonal antibody protection group and an IgG control group, on which corresponding pretreatment was carried out. 24 h later, the Aβ42 oligomers were added to exert an action for 8 h, and cell viability was detected with a CCK8 kit. The results showed that the cell viability of the antibody protection group was significantly higher than those of the cell control group and the IgG control group (P<0.05).

Although the specific embodiments of the present invention are described above in combination with the accompanying drawings, they are not intended to limit the protection scope of the present invention. Based on the technical solutions disclosed by the present invention, those skilled in the art can make a variety of modifications and variations without creative labor, and these modifications and variations should be included within the protection scope of the present invention.

What is claimed is:

1. A composition for detecting an amyloid, comprising a monoclonal antibody AB7G, or a fragment thereof, against human amyloid-β Aβ42, and a monoclonal antibody AB11A2 or a fragment thereof for detecting an Aβ42 monomer or an aggregate thereof in combination with the monoclonal antibody AB7G or the fragment thereof, wherein the monoclonal antibody AB7G or the fragment thereof is able to specifically bind to an Aβ42 monomer and an Aβ42 aggregate; a heavy chain variable region of the monoclonal antibody AB7G has the amino acid sequence of SEQ ID NO: 2; and a light chain variable region of the monoclonal antibody AB7G has the amino acid sequence of SEQ ID NO: 4.

2. The monoclonal antibody composition according to claim 1, wherein a heavy chain variable region of the monoclonal antibody AB11A2 has the amino acid sequence of SEQ ID NO: 6; and a light chain variable region of the monoclonal antibody AB11A2 has the amino acid sequence of SEQ ID NO: 8.

3. The monoclonal antibody composition according to claim 1, wherein a nucleic acid A encoding the heavy chain variable region of the monoclonal antibody AB7G has the DNA sequence of SEQ ID NO: 1; and a nucleic acid B encoding the light chain variable region of the monoclonal antibody AB7G has the DNA sequence of SEQ ID NO: 3.

4. The monoclonal antibody composition according to claim 2, wherein a nucleic acid C encoding the heavy chain variable region of the monoclonal antibody AB11A2 has the DNA sequence of SEQ ID NO: 5; and a nucleic acid D encoding the light chain variable region of the monoclonal antibody AB11A2 has the DNA sequence of SEQ ID NO: 7.

5. The monoclonal antibody composition according to claim 3, wherein the nucleic acid A and B are integrated into expression cassettes or vectors which are transformed into host cells, or integrated into the genomics of the host cells directly.

6. The monoclonal antibody composition according to claim 4, wherein the nucleic acid C and D are integrated into expression cassettes or vectors which are transformed into host cells, or integrated into the genomics of the host cells directly.

7. The monoclonal antibody composition according to claim 1, wherein the monoclonal antibody AB7G or the fragment thereof is conjugated into a complex or a conjugate.

8. The monoclonal antibody composition according to claim 7, wherein the complex is derived by chemically or biologically labeling the monoclonal antibody AB7G or the fragment thereof; and the conjugate is derived by conjugating the monoclonal antibody AB7G or the fragment thereof or the complex to a solid or semi-solid medium.

9. The monoclonal antibody composition according to claim 2, wherein the monoclonal antibody AB11A2 or the fragment thereof is conjugated into a complex or a conjugate.

10. The monoclonal antibody composition according to claim 9, wherein the complex is derived by chemically or biologically labeling the monoclonal antibody AB11A2 or the fragment thereof; and the conjugate is derived by conjugating the monoclonal antibody AB11A2 or the fragment thereof or the complex to a solid or semi-solid medium.

11. A method for treating, preventing and diagnosing Alzheimer's disease comprising a step of administering the monoclonal antibody AB7G or AB11A2 or the fragments thereof of claim 1 to a subject in need wherein the Alzheimer's disease is an Aβ42-driven amyloidosis.

12. A kit for specific quantitative detection of an Aβ42 monomer or an aggregate thereof in human body fluids, comprising: a monoclonal antibody AB7G or the fragment, for capturing the Aβ42 monomer or the aggregate thereof; and a monoclonal antibody AB11A2 or the fragment thereof, for detecting the Aβ42 monomer or the aggregate thereof; wherein the monoclonal antibody AB7G or the fragment thereof is able to specifically bind to an Aβ42 monomer and an Aβ42 aggregate; a heavy chain variable region of the monoclonal antibody AB7G has the amino acid sequence of SEQ ID NO: 2; and a light chain variable region of the monoclonal antibody AB7G has the amino acid sequence of SEQ ID NO: 4; a heavy chain variable region of the monoclonal antibody AB11A2 has the amino acid sequence of SEQ ID NO: 6; and a light chain variable region of the monoclonal antibody AB11A2 has the amino acid sequence of SEQ ID NO: 8.

13. The kit according to claim 12, wherein the monoclonal antibody AB7G or the fragment thereof is fixed on a microplate, and the monoclonal antibody AB11A2 or the fragment thereof is labeled with biotin.


SEQUENCE LISTING (25 sequences)

SEQ ID NO: 1 - DNA, 474 bp, synthetic construct
  source 1..474: mol_type = other DNA
atgagcactgaacacagacacctcaccatgaacttcgggttcagcttgattttccttgtccttgttttaaaaggtgtccagtgtgaagtgaagctggtggagtctgggggaggcttagtgaggcctggagggtccctgaaactctcctgtgcagcctctggattcactttcagtagctatgccatgtcttgggttcgccagactccaaaaaaaaggctggagtgggtcgcatccattagtattagtggtaacgcctactatccaaacaatgtgaagggccgattcaccatctccagagatgatgccaggaacatcctgtacctgcaaatgagcagtctgaggtctgaggacacggccatgtattactgtggaaaaggccacgaagccaactacccctggtttgtttactggggccaagggactctggtcactgtctctgcagctacaacaacagccccatctgtctatcccttg

SEQ ID NO: 2 - protein, 158 aa, synthetic construct
MSTEHRHLTMNFGFSLIFLVLVLKGVQCEVKLVESGGGLVRPGGSLKLSCAASGFTFSSYAMSWVRQTPKKRLEWVASISISGNAYYPNNVKGRFTISRDDARNILYLQMSSLRSEDTAMYYCGKGHEANYPWFVYWGQGTLVTVSAATTTAPSVYPL

SEQ ID NO: 3 - DNA, 384 bp, synthetic construct
  source 1..384: mol_type = other DNA
atggagacagacacactcctgctacgggtactgctgctctgggttccaggttccaccggtgacattgtgctgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaa

SEQ ID NO: 4 - protein, 128 aa, synthetic construct
METDTLLLRVLLLWVPGSTGDIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 5 - DNA, 447 bp, synthetic construct
  source 1..447: mol_type = other DNA
atgaatgattacgattcgagctcggtacccggggatcctctagagattcaggtgcagctgcagaagtctggggctgagctggcaagacctggggcttcagtgaagttgtcctgcaaggcttctggctacacctttactagctactggatgcagtgggtaaaacagaggcctggacagggtctggaatggattggggctatttatcctggagatggtgatactaggtacactcagaagttcaagggcaaggccacattgactgcagataaatcctccagcacagcctacatgcaactcagcagcttggcatctgaggactctgcggtctattactgtgcaagatggggattactacggcactatggtatggactactggggtcaaggaacctcagtcaccgtctcctcagccaaaacaacacccccatcggtctatccactgaatcgt

SEQ ID NO: 6 - protein, 149 aa, synthetic construct
MNDYDSSSVPGDPLEIQVQLQKSGAELARPGASVKLSCKASGYTFTSYWMQWVKQRPGQGLEWIGAIYPGDGDTRYTQKFKGKATLTADKSSSTAYMQLSSLASEDSAVYYCARWGLLRHYGMDYWGQGTSVTVSSAKTTPPSVYPLNR

SEQ ID NO: 7 - DNA, 348 bp, synthetic construct
  source 1..348: mol_type = other DNA
caggtgcagctgcagaagtctgggcctcagctggttaggcctggggcttcagtgaagatatcctgcaaggcttctggttactcattcaccagcttctggatgcactgggtgaagcagaggcctggacaaggtcttgagtggattggcatgattgatccttccgatagtgaaactaggttaaatcagaagttcaaggacaaggcctcattgactgtagacgaatattccagcacagcctacatgcaactcagcagcccgacatctgaggactctgcggtctattactgtgcaagaatgggatggttggggaactactggggtcaaggaacctcagtcaccgtctcctca

SEQ ID NO: 8 - protein, 116 aa, synthetic construct
QVQLQKSGPQLVRPGASVKISCKASGYSFTSFWMHWVKQRPGQGLEWIGMIDPSDSETRLNQKFKDKASLTVDEYSSTAYMQLSSPTSEDSAVYYCARMGWLGNYWGQGTSVTVSS

SEQ ID NO: 9 - protein, 6 aa, synthetic construct
GGVVIA

SEQ ID NO: 10 - protein, 8 aa, synthetic construct
LVFFAEDV

SEQ ID NO: 11 - DNA, 19 bp, synthetic construct
  source 1..19: mol_type = other DNA
ctggatggtgggaagatgg

SEQ ID NO: 12 - DNA, 21 bp, synthetic construct
  source 1..21: mol_type = other DNA
caagggatagacagatggggc

SEQ ID NO: 13 - DNA, 30 bp, synthetic construct
  source 1..30: mol_type = other DNA
ggccacgcgtcgactagtacgggggggggg

SEQ ID NO: 14 - DNA, 30 bp, synthetic construct
  source 1..30: mol_type = other DNA
atggatttwcaggtgcagattwtcagcttc

SEQ ID NO: 15 - DNA, 21 bp, synthetic construct
  source 1..21: mol_type = other DNA
cagtggatagaccgatggggg

SEQ ID NO: 16 - DNA, 20 bp, synthetic construct
  source 1..20: mol_type = other DNA
actggatggtgggaagatgg

SEQ ID NO: 17 - DNA, 30 bp, synthetic construct
  source 1..30: mol_type = other DNA
atggagwcagacacactcctgytaygggtg

SEQ ID NO: 18 - DNA, 1434 bp, synthetic construct
  source 1..1434: mol_type = other DNA
atgagcactgaacacagacacctcaccatgaacttcgggttcagcttgattttccttgtccttgttttaaaaggtgtccagtgtgaagtgaagctggtggagtctgggggaggcttagtgaggcctggagggtccctgaaactctcctgtgcagcctctggattcactttcagtagctatgccatgtcttgggttcgccagactccaaaaaaaaggctggagtgggtcgcatccattagtattagtggtaacgcctactatccaaacaatgtgaagggccgattcaccatctccagagatgatgccaggaacatcctgtacctgcaaatgagcagtctgaggtctgaggacacggccatgtattactgtggaaaaggccacgaagccaactacccctggtttgtttactggggccaagggactctggtcactgtctctgcagctacaacaacagccccatctgtctatcccttggtgcctggctgttctgacacctctggctcctctgtgaccctgggctgtctggtgaagggctactttcctgaacctgtgacagtgaagtggaactatggagccctgtcctctggagtgaggacagtgtcctctgtgctccaatctggcttctactccctgtcctccctggtgacagtgccatccagcacctggccaagccagacagtgatttgtaatgtggctcaccctgccagcaagacagaactgattaagaggattgaaccaaggattccaaagccaagcacacctcctggctcctcctgtcctcctggcaacatcctgggaggaccatctgtgttcatctttcctccaaagccaaaggatgccctgatgatttccctgacaccaaaggtgacttgtgtggtggtggatgtgtctgaggatgaccctgatgtgcatgtgtcctggtttgtggacaacaaggaggtgcatacagcctggacccaaccaagggaggctcaatacaacagcaccttcagggtggtgtctgccctgccaatccaacaccaggactggatgaggggcaaggagttcaagtgtaaggtgaacaacaaggctctgcctgccccaattgagaggaccatcagcaagccaaagggcagggctcaaacaccacaggtctacaccatccctcctccaagggaacagatgagtaagaagaaggtgtccctgacttgtctggtgaccaacttcttctctgaggctatctctgtggagtgggagaggaatggagaattggaacaggactacaagaacacacctccaatcctggactctgatggcacctacttcctctacagcaaactgacagtggacacagactcctggctccaaggagagattttcacttgttctgtggtccatgaggctctgcacaaccaccacacccagaagaacctgagcaggagccctggcaagtga

SEQ ID NO: 19 - protein, 477 aa, synthetic construct
MSTEHRHLTMNFGFSLIFLVLVLKGVQCEVKLVESGGGLVRPGGSLKLSCAASGFTFSSYAMSWVRQTPKKRLEWVASISISGNAYYPNNVKGRFTISRDDARNILYLQMSSLRSEDTAMYYCGKGHEANYPWFVYWGQGTLVTVSAATTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPGK

SEQ ID NO: 20 - DNA, 708 bp, synthetic construct
  source 1..708: mol_type = other DNA
atggagacagacacactcctgctacgggtactgctgctctgggttccaggttccaccggtgacattgtgctgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttaa

SEQ ID NO: 21 - protein, 235 aa, synthetic construct
METDTLLLRVLLLWVPGSTGDIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 22 - DNA, 1398 bp, synthetic construct
  source 1..1398: mol_type = other DNA
atgaatgattacgattcgagctcggtacccggggatcctctagagattcaggtgcagctgcagaagtctggggctgagctggcaagacctggggcttcagtgaagttgtcctgcaaggcttctggctacacctttactagctactggatgcagtgggtaaaacagaggcctggacagggtctggaatggattggggctatttatcctggagatggtgatactaggtacactcagaagttcaagggcaaggccacattgactgcagataaatcctccagcacagcctacatgcaactcagcagcttggcatctgaggactctgcggtctattactgtgcaagatggggattactacggcactatggtatggactactggggtcaaggaacctcagtcaccgtctcctcagccaaaacaacacccccatcggtctatccactgaatcgtgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 23 - protein, 466 aa, synthetic construct
MNDYDSSSVPGDPLEIQVQLQKSGAELARPGASVKLSCKASGYTFTSYWMQWVKQRPGQGLEWIGAIYPGDGDTRYTQKFKGKATLTADKSSSTAYMQLSSLASEDSAVYYCARWGLLRHYGMDYWGQGTSVTVSSAKTTPPSVYPLNRVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 24 - DNA, 669 bp, synthetic construct
  source 1..669: mol_type = other DNA
caggtgcagctgcagaagtctgggcctcagctggttaggcctggggcttcagtgaagatatcctgcaaggcttctggttactcattcaccagcttctggatgcactgggtgaagcagaggcctggacaaggtcttgagtggattggcatgattgatccttccgatagtgaaactaggttaaatcagaagttcaaggacaaggcctcattgactgtagacgaatattccagcacagcctacatgcaactcagcagcccgacatctgaggactctgcggtctattactgtgcaagaatgggatggttggggaactactggggtcaaggaacctcagtcaccgtctcctcaagggctgacgccgctcctaccgtgtccatcttccctcctagctccgagcagctgaccagcggcggcgctagcgtggtgtgttttctgaataacttttaccctaaggatatcaacgtgaagtggaagatcgatggcagcgagaggcagaacggcgtgctgaatagctggaccgatcaggattccaaggacagcacctacagcatgagcagcacactgacactgaccaaggacgagtacgagagacacaattcctacacctgtgaggccacccacaagaccagcacctcccctatcgtgaagagctttaacaggaacgagtgc

SEQ ID NO: 25 - protein, 223 aa, synthetic construct
QVQLQKSGPQLVRPGASVKISCKASGYSFTSFWMHWVKQRPGQGLEWIGMIDPSDSETRLNQKFKDKASLTVDEYSSTAYMQLSSPTSEDSAVYYCARMGWLGNYWGQGTSVTVSSRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC